(19) United States
(12) Patent Application Publication
(10) Pub. No.: US 2013/0236507 A1
(43) Pub. Date: Sep. 12, 2013

(54) Method for treating cancer by using Fe-containing alloy particles

(71) Applicant: NATIONAL CHENG KUNG UNIVERSITY, Tainan City (TW)

(72) Inventors: Dar-Bin SHIEH, Tainan City (TW); Li-Xing YANG, New Taipei City (TW); Ya-Na WU, Yunlin County (TW)

(73) Assignee: NATIONAL CHENG KUNG UNIVERSITY, Tainan City (TW)

(21) Appl. No.: 13/795,571

(22) Filed: Mar. 12, 2013

Related U.S. Application Data
(60) Provisional application No. 61/609,430, filed on Mar. 12, 2012.

(51) Int. Cl. A61K 33/26; A61K 9/14; A61K 33/38

(52) U.S. Cl. CPC A61K 33/26; A61K 33/38; A61K 9/14; Y10S 977/773; Y10S 977/81; B82Y 5/00
USPC 424/400; 424/646; 424/618; 977/773; 977/810; 977/915

(57) ABSTRACT

A method for treating a cancer is disclosed, which comprises: administering an effective amount of Fe-containing alloy particles to a subject in need, wherein a material of each Fe-containing alloy particle is an alloy comprising a first metal of Fe and a second metal.

### CROSS REFERENCE TO RELATED APPLICATION

This application claims the benefit of filing date of U.S. Provisional Application Ser. No. 61/609,430, entitled “Application of non-oxidized Fe containing alloy particles for tumor therapeutics” filed Mar. 12, 2012 under 35 USC §119(e)(1).

### BACKGROUND OF THE INVENTION

1. Field of the Invention

The present invention relates to a method for treating a cancer and, more particularly, to a method for treating a cancer by using Fe-containing alloy particles.

2. Description of Related Art

Foods or food additives, and environmental pollutions have been a source of contention as a cause or catalyst for promoting cancer in recent years. Not coincidentally, the same event is happening as well in the developed countries and around the world, positing as an alarming sign that the incidence rates of cancers are quite high. According to the data published by the American Cancer Society, cancer is being proved to be the most significant threat to public health.

The general methods for treating cancer include surgery, radiotherapy, chemotherapy and immune therapy. In recent years, the development of several therapeutic agents has led to cancer treatments through new anti-cancer mechanisms, and it has been proven that the survival rate of patients can be increased by treating them with these therapeutic agents. Generally, the therapeutic agents can treat cancers through inhibition of cell cycle progression, angiogenesis, farnesyl transferase, and tyrosine kinases. Although it is known that certain agents exhibit therapeutic effects on cancer, these agents do have their limitations. For example, the commercial chemical agents kill not only tumor cells but also normal cells. Hence, it is desirable to provide novel anti-cancer agents which can only kill tumor cells but keep normal cells survive.

On the other hands, nanoparticles have unique electrical, chemical, physical and optical properties due to its size associated effects. Organic or inorganic nanoparticles have been applied as carriers to transport and deliver drugs or genes into target organs or cells. Alternatively, some nanoparticles could convert externally applied energy to therapeutics such as hyperthermia, free radical generation and ionic radiation. Since the nanoparticles have the aforementioned properties, the applications thereof can further be increased if it is improved that the nanoparticles has anti-cancer properties.

### SUMMARY OF THE INVENTION

The object of the present invention is to provide a method for treating a cancer, wherein the Fe-containing alloy particles used herein can selectively kill tumor cells without significant cytotoxicity to normal cells.

To achieve the object, the method for treating the cancer of the present invention comprises: administering an effective amount of Fe-containing alloy particles to a subject in need, wherein a material of each Fe-containing alloy particle is an alloy comprising a first metal of Fe and a second metal.

In the method for treating the cancer of the present invention, the Fe-containing alloy particles can be used as a self-detoxification anti-cancer drug. When the Fe-containing alloy particles of the present invention are applied to the cancer treatment, an effective amount thereof can selectively kill tumor cells and inhibit the growth of the tumor cells. In addition, the Fe-containing alloy particles of the present invention can selectively kill tumor cells and show no significant adverse effects to normal cells. Hence, when the Fe-containing alloy particles are applied on tumor therapies, the first metal of Fe contained in the alloy particles can spontaneously be oxidized after the accomplishment of the anti-cancer mission, and the side effect thereof can further be reduced. In addition, the Fe-containing alloy particles of the present invention can also be used as diagnostic reagents, such as imaging agents for Magnetic Resonance (MRI) or Computed Tomography (CT). Hence, when the Fe-containing alloy particles are applied to the subject in need, the tracing object of the Fe-containing alloy particles can further be achieved. Therefore, the Fe-containing alloy particles of the present invention have dual-functions of anti-cancer drugs and imaging agents.

In the method of the present invention, the subject can be mammalian. Preferably, the subject is human.

In the method of the present invention, the cancer can be any types of cancers generally known in the art, such as bladder cancer, bone cancer, brain cancer, breast cancer, cervical cancer, colon cancer, endometrial cancer, esophageal cancer, leukemia, liver cancer, lymphoma, kidney cancer, osteosarcoma, ovarian cancer, pancreatic cancer, prostate cancer, skin cancer including basal and squamous cell carcinoma and melanoma, small intestine cancer, stomach cancer, thymus cancer and thyroid cancer, but the scope of applicability of the present invention is not limited thereto. Preferably, the cancer is an oral cancer, which may be classified into various histological types such as teratoma, adenocarcinoma derived from a major or minor salivary gland, lymphoma from tonsillar or other lymphoid tissue, or melanoma from the pigment-producing cells of the oral mucosa.

In the method of the present invention, the first metal of Fe is Fe with zero valences. In addition, the second metal can be at least one selected from the group consisting of Ag, Pt, and Au. Preferably, the second metal is Au. The specific examples of each Fe-containing alloy particles may comprise FeAg particles, FePt particles, or FeAu particles. Most preferably, the Fe-containing alloy particles are FeAu particles.

Furthermore, in the method of the present invention, a size of each Fe-containing alloy particle is respectively in a nano- or submicro-scale. For example, the size of each Fe-containing alloy particle is respectively in a range from 2 nm to 5 μm. Preferably, the size of each Fe-containing alloy particle is respectively in a range from 2 nm to 1 μm. More preferably, the size of each Fe-containing alloy particle is respectively in a range from 2 nm to 50 nm.

Moreover, in the method of the present invention, a shape of each Fe-containing alloy particle is not particularly limited, as along as the Fe-containing alloy particle as the aforementioned size. For example, the shape of each Fe-containing alloy particle can be a rod, a sphere, a cubic or a dumbbell. Preferably, the shape of each Fe-containing alloy particle is a sphere or a cubic.

Except for the aforementioned aspects of the method for treating cancers, the present invention further provides a pharmaceutical composition for tumor therapeutics, which comprises an effective amount of the aforementioned Fe-containing alloy particles; and a pharmaceutically acceptable carrier.

The Fe-based particles and the pharmaceutical composition for treating cancers of the present invention can be administered via parenteral, inhalation, local, rectal, nasal, sublingual, or vaginal delivery, or implanted reservoir. Herein, the term “parenteral delivery” includes subcutaneous, intradermic, intravenous, intra-articular, intra-arterial, synovial, intrapleural, intrathecal, local, and intracranial injections.

According to the pharmaceutical composition of the present invention, the term “pharmaceutically acceptable carrier” means that the carrier must be compatible with the active ingredients (and preferably, capable of stabilizing the active ingredients) and not be deleterious to the subject to be treated. The carrier may be at least one selected from the group consisting of active agents, adjuvants, dispersants, wetting agents and suspending agents. The example of the carrier may be microcrystalline cellulose, mannitol, glucose, non-fat milk powder, polyethylene, polyvinylprrolidone, starch or a combination thereof.

In addition, the term “treating” used in the present invention refers to the application or administration of the Fe-based particles or the pharmaceutical composition containing the Fe-based particles to a subject with symptoms or tendencies of suffering from cancer in order to cure, heal, alleviate, relieve, alter, remedy, ameliorate, improve, prevent or affect the symptoms or tendencies of cancers. Furthermore, “an effective amount” used herein refers to the amount of each active ingredients such as the Fe-based particles required to confer therapeutic effect on the subject. The effective amount may vary according to the route of administration, excipient usage, and co-usage with other active ingredients.

Other objects, advantages, and novel features of the invention will become more apparent from the following detailed description when taken in conjunction with the accompanying drawings.

### BRIEF DESCRIPTION OF THE DRAWINGS

FIG. 1 is a result showing the cytotoxicity of FeAu alloy particles according to Embodiment 1 of the present invention;

FIG. 2 is a result showing the cytotoxicity of FeAu alloy particles according to Embodiment 1 of the present invention when exposing to the air; and

FIG. 3 is a result showing the cytotoxicity of Fe alone particles freshly prepared and stored in −20° C. for 6 months.

### DETAILED DESCRIPTION OF THE PREFERRED EMBODIMENT

The present invention has been described in an illustrative manner, and it is to be understood that the terminology used is intended to be in the nature of description rather than of limitation. Many modifications and variations of the present invention are possible in light of the above teachings. Therefore, it is to be understood that within the scope of the appended claims, the invention may be practiced otherwise than as specifically described.

### [Material and Method]

### Embodiment 1

### Preparation of FeAu Alloy Particles

Iron pentacarbonyl (50 mM), gold acetate (12.5 mM), and 1,2-hexadecanediol (75 mM) were dissolved in dioctylether solution with oleic acid (25 mM) and oleyl amine (25 mM) to a final volume of a 20 mL in Argon. After stirring for 1 h at room temperature, the temperature was then raised to 250° C., and the reaction mixture was refluxed for 1 h under Argon flow to form FeAu particles. Subsequently, the reaction mixture was cooled to room temperature and precipitated by addition of 40 mL of ethanol. The precipitation was then recovered by a magnet and then washed for 3 times with hexane and ethanol. Finally, the washed particles were dried under vacuum. After the aforementioned process, Fe-containing alloy particles (i.e. FeAu alloy particles) were obtained. Herein, the size of each FeAu alloy particle is about 4.90±1.16 nm, and the shape thereof is in sphere.

### Cytotoxicity Assay (MTT Assay)

To evaluate the cytotoxicity of the obtained particles, cells growing in log-phase were seeded at a density of 5,000 cells per well in a 96-well culture plate. Prior to each experiment for administration in vitro, particles obtained in Embodiment 1 were resuspended in phosphate buffered saline solution (PBS) at 10 mg/mL as the stock solution. Cells were then treated by the assigned concentration for 48 hours. The 10×MTT stock (5 mg/mL) in PBS was diluted with culture medium as working solution. Cells were treated with the working solution for 1 hour. Later, the working solution was replaced by 50 μL DMSO to dissolve the violet crystal. The optical absorbance at 490 nm was measured in a microplate reader (Sunrise Absorbance Reader; Tecan, Minnedorf, Switzerland). The cell viability was defined as: (O.D._{treated cells}−O.D._{DMSO blank})/(O.D._{untreated cells}−O.D._{DMSO blank})*100%

### [Results]

### Evaluation on the Cytotoxicity of FeAu Alloy Particles Prepared in Embodiment 1

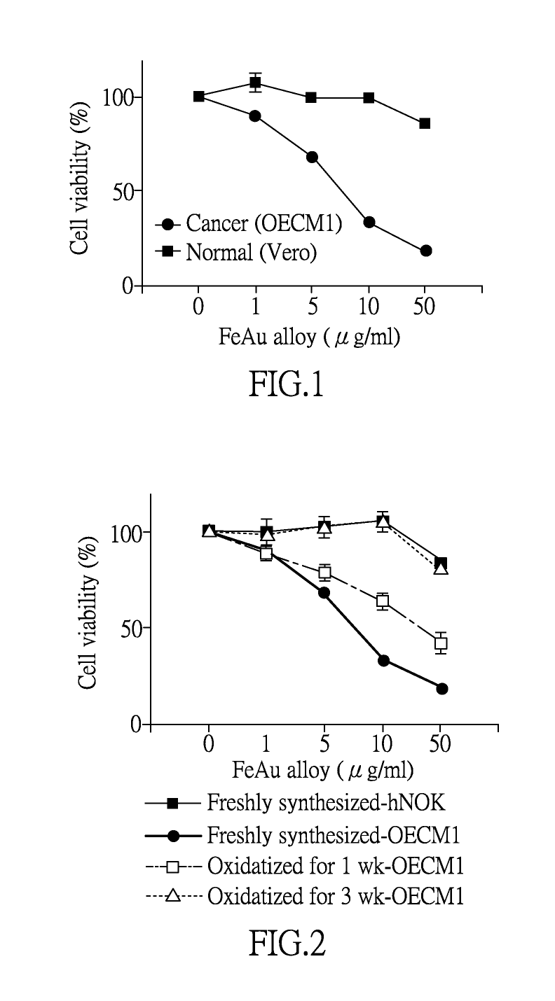

The cytotoxicity of FeAu alloy particles on OECM1 cell lines (tumor cells) and Vero cell lines (normal cells) was respectively also evaluated by the aforementioned MTT assay. The result is shown in the FIG. 1.

The X-axis of FIG. 1 is the addition amount of FeAu alloy particles, and the Y-axis thereof is the cell viability of cancer cells and normal cells treated with the FeAu alloy particles, in which the cell viability that the cells were not treated with the FeAu alloy particles was considered as 100%. As shown in FIG. 1, when the cancer cells are treated with FeAu alloy particles, the cell viability of cancer cells is about 25% at the dosage of 10 μg/mL. However, even though the dose of the FeAu alloy particles is as much as 50 μg/mL, the cell viability of normal cells is still about 100%. This result indicates that the FeAu alloy particles prepared in Embodiment 1 show killing selectivity to cancer cells, while sparing most of the normal cells.

The Aging Process of the Cytotoxicity of FeAu Alloy Prepared in Embodiment 1 when Exposing to the Air

The zero valent iron in FeAu alloy particle is proposed as the key factor to kill cancer. Therefore, the anti-cancer property of freshly prepared FeAu alloy particles was compared with those exposed to air in a period of time by the aforementioned MTT assay. Herein, OECM 1 cell lines (cancer cells) and hNOK cell lines (normal cells) were used. The result is shown in FIG. 2, wherein the X-axis thereof is the addition amount of FeAu alloy particles, and the Y-axis thereof is the cell viability of cancer cells and normal cells treated with the FeAu alloy particles, in which the cell viability that the cells were not treated with the FeAu alloy particles was considered as 100%.

As shown in FIG. 2, the freshly synthesized show killing selectivity to cancer cells, while sparing most of the normal cells; and this result is consistent with that shown in FIG. 1. In addition, as shown in FIG. 2, the anticancer effect of FeAu alloy particles was diminished with the increase of time period exposing to the air. This result indicates that the zero valent iron in FeAu alloy particle is a key factor to kill cancer. Therefore, the synthesized FeAu alloy particles have to be stored in a proper condition to keep the zero valence of Fe in the FeAu alloy particles, in order to maintain the anti-cancer activity thereof.

According to the results of FIG. 1 and FIG. 2, the FeAu alloy particles show selective anti-cancer effect and the spontaneously detoxify themselves after accomplish their anticancer mission.

The Storage of Fe-Containing Alloy Particles and their Preservation

Since the fast detoxification effect of Fe-containing alloy particles may affect the practicality of the clinical application, a proper storage condition therefor is necessary to facilitate the clinical usage of Fe-containing alloy particles as well as to lower the potential cost. The oxidations of Fe are including the anaerobic corrosion (1) and Aerobic corrosion (2).

Fe₍₀₎+2H₂O→Fe²⁺+H₂+2OH⁻ (1)

2Fe₍₀₎+O₂+2H₂O→2Fe²⁺+4OH⁻ (2)

It has been well known that water is essential for the corrosion of Fe. Therefore, in the present embodiment, the obtained FeAu alloy particles were stored in oxygen-depleted ethanol in order to prevent the oxidation of Fe particles.

Here, the OECM 1 cell lines (tumor cells) were treated with Fe alone particles to identify the cytotoxicity thereof. The Fe alone particles were synthesized as follows. 20 mL 1-octadecene and 0.3 mL oleylamine was mixed at 120° C. for 30 min in argon. Later, 0.7 mL iron pentacarbonyl was added to the 1-octadecene and oleylamine mixture at 180° C. for 20 min with continuously argon condition. The solution was cooled down to 160° C. before the further addition of 0.15 mL iron pentacarbonyl. Then, the solution was aged at 160° C. for 30 min with the presence of 0.3 ml oleic acid (1 mM). The particles were then washed with hexane and ethanol, and then kept in argon prior to the use.

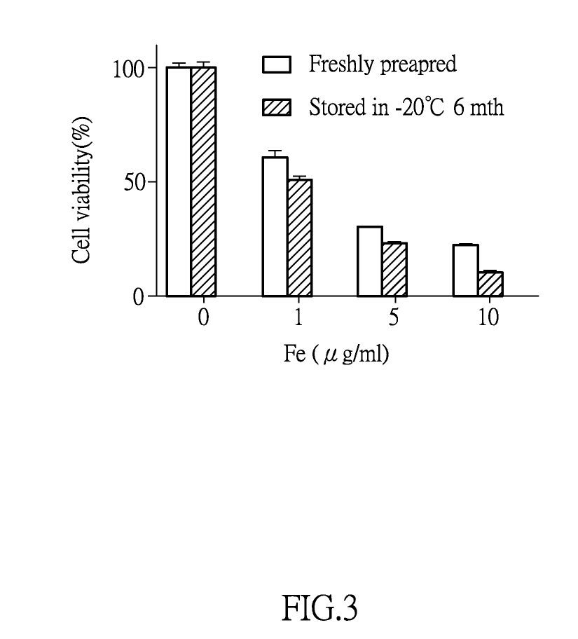

The obtained Fe alone particles with a size of 15.34±1.39 nm and a sphere shape were examined by using the aforementioned MTT assay, and the result is shown in FIG. 3, wherein the X-axis thereof is the addition amount of Fe alone particles, and the Y-axis thereof is the cell viability of cancer cells treated with the Fe alone particles, in which the cell viability that the cells were not treated with the Fe alone particles was considered as 100%.

As shown in FIG. 3, the Fe alone particles stored in oxygen-depleted ethanol in freezer (−20° C.) shows similar cell viability to the Fe alone particles prepared freshly. This result indicates that a suitable storing condition to the Fe particle can effectively preserve the cytotoxicity of Fe particles.

In conclusion, the Fe-containing alloy particles without any modification of the present invention show great killing selectivity to tumor cells. In addition, when the Fe-containing alloy particles stored in a suitable environment, for example cold ethanol or liquid with low reactivity, the activity of the Fe-containing alloy particles can be maintained for a long time. Hence, the Fe-containing alloy particles of the present invention can be prepared in advance, and then applied to tumor therapeutics. In addition, the Fe-containing alloy particles also have magneticity, so they can be applied to various diagnoses. For example, the FeAg alloy particles show non-linear optical frequency multiplication, and have potential on medical imaging application. Furthermore, the FePt alloy particles can also be used as contrast agents for CT imaging. Hence, Fe-containing alloy particles of the present invention can be modulated based on the patient's symptoms, the applications, the diagnosis methods, and the therapeutic methods.

Although the present invention has been explained in relation to its preferred embodiment, it is to be understood that many other possible modifications and variations can be made without departing from the spirit and scope of the invention as hereinafter claimed.

What is claimed is:

1. A method for treating a cancer, comprising:
administering an effective amount of Fe-containing alloy particles to a subject in need, wherein a material of each Fe-containing alloy particle is an alloy comprising a first metal of Fe and a second metal.

2. The method as claimed in claim 1, wherein the cancer is an oral cancer.

3. The method as claimed in claim 1, wherein the first metal of Fe is Fe with zero valences.

4. The method as claimed in claim 1, wherein the second metal is at least one selected from the group consisting of Ag, Pt, and Au.

5. The method as claimed in claim 1, wherein each Fe-containing alloy particles is respectively a FeAg particle, a FePt particle, or a FeAu particle.

6. The method as claimed in claim 1, wherein a size of each Fe-containing alloy particle is respectively in a range from 2 nm to 5 μm.

7. The method as claimed in claim 6, wherein the size of each Fe-containing alloy particle is respectively in a range from 2 nm to 1 μm.

8. The method as claimed in claim 7, wherein the size of each Fe-containing alloy particle is respectively in a range from 2 nm to 50 nm.

9. The method as claimed in claim 1, wherein a shape of each Fe-containing alloy particle is a rod, a sphere, a cubic or a dumbbell.
